(19) United States
(12) Patent Application Publication
(10) Pub. No.: US 2026/0013713 A1
(43) Pub. Date: Jan. 15, 2026

(54) MEDICAL DEVICES, SYSTEMS, AND RELATED METHODS FOR ILLUMINATING A TREATMENT SITE

(71) Applicant: Lumenis Ltd., Yokneam (IL)

(72) Inventor: Gassan DIAB, Haifa (IL)

(73) Assignee: Lumenis Ltd., Yokneam (IL)

(21) Appl. No.: 19/262,360

(22) Filed: Jul. 8, 2025

Related U.S. Application Data
(60) Provisional application No. 63/669,352, filed on Jul. 10, 2024.

(51) Int. Cl. A61B 1/307; A61B 1/00; A61B 1/015; A61B 1/06

(52) U.S. Cl. CPC A61B 1/307; A61B 1/00066; A61B 1/00082; A61B 1/00085; A61B 1/00087; A61B 1/00135; A61B 1/015; A61B 1/06

(57) ABSTRACT

Medical systems including a medical device are described. The medical device may include a shaft defining working channels, wherein each working channel has a respective lumen with a respective distal opening and the distal openings are positioned at a distal portion of the shaft. The medical device may further include an imaging device positioned at the distal portion of the shaft. The medical system may further include an instrument movable through one or more of the working channels; and a light source. A distal portion of the instrument may include an expandable end effector. The light source may be releasably positionable within the expandable end effector of the instrument.

### CROSS-REFERENCE TO RELATED APPLICATION(S)

This application claims the benefit of priority to U.S. Provisional Application No. 63/669,352, filed on Jul. 10, 2024, which is incorporated by reference herein in its entirety.

### TECHNICAL FIELD

Aspects of this disclosure generally relate to medical systems, devices, and methods related to treating a subject using a scope, such as an ureteroscope, and illuminating at least a portion of a treatment site.

### BACKGROUND

Scopes may be used to treat various medical conditions. For example, ureteroscopes may be used to view and/or treat kidney stones or portions of the urinary tract. As such, ureteroscopes include a working channel through which an instrument may be inserted. Examples of instruments include, but are not limited to, baskets, snares, ablation tools, fibers, and so forth. Ureteroscopes may further include a light source for illumination and/or an imaging device to view inside a lumen of the body (e.g., a portion of a kidney).

An outside diameter (and thus a cross-sectional area) of a ureteroscope is constrained due to a typical size of a urethra. Further, one or more portions of the shaft of the ureteroscope is often flexible to enable movement within the confines of the urethra and urinary tract. Therefore, the outside diameter and material properties of the ureteroscope impact utility, flexibility, and performance. Additionally, a diameter of the working channel may affect the types and/or sizes of instruments that may be used. Given these constraints, more efficient use of the cross-sectional area of the ureteroscope is desired.

### SUMMARY

In some aspects, the techniques described herein relate to a medical system including: a medical device including: a shaft defining working channels, each working channel having a respective lumen with a respective distal opening, the distal openings positioned at a distal portion of the shaft; and an imaging device positioned at the distal portion of the shaft; an instrument movable through one or more of the working channels; and a light source. A distal portion of the instrument includes an expandable end effector. The light source is releasably positionable within the expandable end effector of the instrument. The medical device does not include a light source.

In some aspects, the techniques described herein relate to a medical system. The medical device further includes a handle positioned on a proximal end of the shaft. The handle includes a port having a corresponding working channel of the working channels.

In some aspects, the techniques described herein relate to a medical system. The instrument does not include an imaging device.

In some aspects, the techniques described herein relate to a medical system. The medical device is a ureteroscope.

In some aspects, the techniques described herein relate to a medical system. The expandable end effector includes an expandable basket.

In some aspects, the techniques described herein relate to a medical system. The light source includes an outer tube and an inner vial. The outer tube contains a first substance and the inner vial contains a second substance. When mixed, the first substance and the second substance cause a chemical reaction that causes a light to be emitted.

In some aspects, the techniques described herein relate to a medical system. The outer tube is formed of polycarbonate plastic. The inner vial is formed of glass.

In some aspects, the techniques described herein relate to a medical system. The light source includes a diameter less than a diameter of at least one of the working channels.

In some aspects, the techniques described herein relate to a medical system. A first working channel of the working channels includes a first diameter. A second working channel of the working channels includes a second diameter that is greater than the first diameter.

In some aspects, the techniques described herein relate to a medical system. The first diameter is within a range of 0.02 to 0.04 inches. The second diameter is within a range of 0.05 to 0.07 inches.

In some aspects, the techniques described herein relate to a medical system. The first diameter is 0.03 inches. The second diameter is 0.06 inches.

In some aspects, the techniques described herein relate to a medical system. The medical device further includes a pressure sensor positioned on the distal portion of the shaft.

In some aspects, the techniques described herein relate to a medical system. The expandable end effector is configurable to releasably retain the light source and position the light source within a lumen of a body.

In some aspects, the techniques described herein relate to a medical system, further including an additional instrument that is movable through an additional working channel of the working channels.

In some aspects, the techniques described herein relate to a medical system. The additional instrument is a laser fiber.

In some aspects, the techniques described herein relate to a method of treatment of a body of a subject, the method including activating a light source; and securing the light source within an expandable end effector of a first instrument. The expandable end effector is positioned at a distal portion of the first instrument and is configured to releasably retain the light source. The method further includes extending a distal portion of the first instrument through a first working channel of a medical device, the medical device including a shaft. The medical device includes the first working channel and a second working channel each extending longitudinally through the shaft. The method further includes introducing the shaft into a lumen of the body; releasing, via the expandable end effector, the light source into the body; and extending a second instrument through the second working channel.

In some aspects, the techniques described herein relate to a method. The expandable end effector is an expandable basket. The method further including, prior to releasing the light source into the body, positioning, with the expandable end effector of the first instrument, the light source into the body by partially extending the first instrument distal to the distal portion of the shaft.

In some aspects, the techniques described herein relate to a method further including: performing a medical treatment with the second instrument; securing, using the end effector of the first instrument, debris resulting from the medical treatment; removing, using the first instrument, the debris via the first working channel; and retrieving, using the first instrument, the light source from the lumen of the body via the first working channel.

In some aspects, the techniques described herein relate to a method of treatment of a body of a subject, the method including activating a light source. The light source includes an outer tube and an inner vial and the outer tube and the inner vial contain substances that mix to emit light. The method further includes securing the light source within an expandable end effector of a first instrument and introducing a shaft of a medical device into and through a lumen of the body. The medical device includes at least a first working channel and a second working channel extending longitudinally through the shaft, and the medical device includes an imaging device at a distal portion of the shaft. The method further includes extending the first instrument through the first working channel of the medical device. The first instrument is configured to releasably retain the activated light source. The method further includes positioning, with the expandable end effector, the activated light source into the body by partially extending the first instrument partially distal to the distal portion of the shaft.

In some aspects, the techniques described herein relate to a method, further including removing the first instrument at least partially through the first working channel; re-inserting the first instrument via the first working channel; capturing the light source with the expandable end effector; and removing the first instrument and the light source via the first working channel.

### BRIEF DESCRIPTION OF THE FIGURES

The accompanying drawings, which are incorporated in and constitute a part of this application, illustrate exemplary aspects that, together with the written descriptions, serve to explain the principles of this disclosure. Each figure depicts one or more exemplary aspects according to this disclosure, as follows:

FIGS. 1A and 1B depict an exemplary medical system according to aspects of this disclosure.

FIG. 2 depicts an exemplary light source, according to aspects of this disclosure.

FIG. 3 depicts a flow chart of an exemplary method for performing a medical procedure, according to aspects of this disclosure.

FIGS. 4A-4H depict views of operation of an exemplary medical system, according to aspects of this disclosure.

### DETAILED DESCRIPTION

Reference will now be made in detail to aspects and examples of this disclosure and illustrated in the accompanying drawings. Wherever possible, the same reference numbers will be used throughout the drawings to refer to the same or like parts.

Both the foregoing general description and the following detailed description are exemplary and explanatory only and are not restrictive of the features, as claimed. As used herein, the terms “comprises,” “comprising,” “has,” “having,” “includes,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

Certain aspects of this disclosure relate to medical devices and systems, such as ureteroscopes, and related methods. Ureteroscopes may be used to treat medical conditions in the body such as kidney stones. For example, a ureteroscope may include a shaft that can be introduced into a body lumen, such as a ureter, and advanced through the urethra until a distal portion of the shaft is located in a calyx of a kidney. The ureteroscope may include one or more working channels through which instruments may be inserted, for example, to treat a kidney stone.

As discussed above, an outside diameter of the shaft of a ureteroscope impacts flexibility and/or performance. Additionally, the diameter of the working channel of the shaft determines a level of functionality of the ureteroscope due to limiting types and/or sizes of instruments that may be used within the working channel.

These constraints present technical challenges for ureteroscopes. Certain aspects address these challenges by removing one or more features from the ureteroscope, such as a light source, and adding one or more additional working channels. Additionally, the shaft of the ureteroscope may be smaller than a shaft of a ureteroscope that includes a light source. The additional working channel(s) may help to improve the functionality and utility of the ureteroscope. Alternatively or additionally, the working channel(s) of the ureteroscope may be larger than the working channel(s) of a ureteroscope that includes a light source.

For example, a ureteroscope may include a light source (e.g., a Light Emitting Diode (LED) light source) positioned at a distal portion of the shaft. The light source may occupy a diameter of Ø0.031 inches (Ø0.7874 mm), representing a cross-sectional area of 0.000754 square inches (0.486 square mm) of the distal portion. By removing the light source, a secondary working channel may be added.

The second working channel may have the same or similar diameter, a greater diameter, or a smaller diameter. The secondary working channel may be approximately or more than half the size of primary working channel, for example, Ø0.03 inches (0.762 mm) in diameter, representing a cross-sectional area of 0.000707 square inches (0.456 square mm). In another example, the primary working channel may have a diameter in the range of 0.02 to 0.04 inches, whereas the second working channel may have a diameter of 0.05 to 0.07 inches. In another example, the primary working channel may have a diameter of 0.03 inches, whereas the second working channel may have a diameter of 0.06 inches. In this manner, two instruments can be used simultaneously. Additionally or alternatively, the shaft of the ureteroscope may be smaller when the distal portion of the shaft does not include a light source and/or the primary working channel may be larger when the distal portion of the shaft does not include a light source.

Relative to existing systems, therefore, disclosed ureteroscopes may help to increase the functionality of the ureteroscope by including two or more working channels. As explained further herein, having an additional working channel may help to increase the functionality of the ureteroscope, for example, by reducing a required number of insertions and removals of various instruments during the procedure. This may help to improve the medical procedure by providing additional flexibility for the physician and/or may help to reduce an amount of time required to complete the procedure.

Because a light source is often necessary for visibility of the inside of the body, certain aspects involve an external light source. In an example, an external light source, typically in the shape of a small pill or capsule, that can be inserted into, positioned, repositioned, and/or removed from a subject by using an instrument, through a working channel as needed.

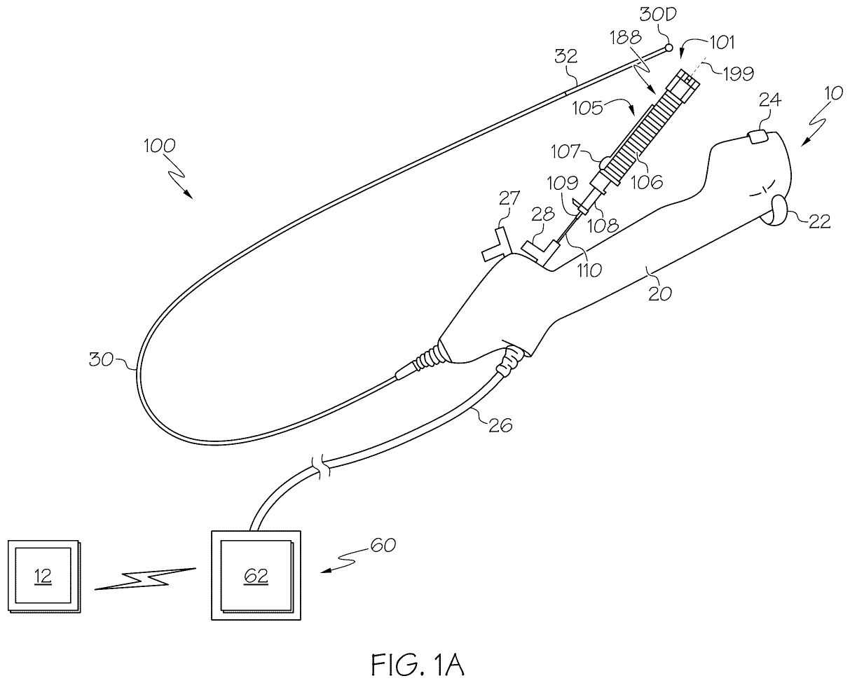

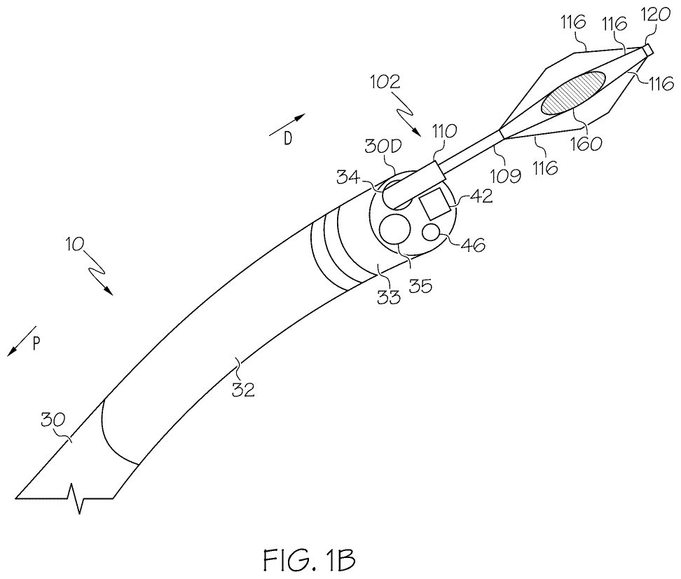

FIGS. 1A and 1B depict an exemplary medical system 100, according to aspects of this disclosure. Medical system 100 includes a medical device 10, which may be coupled to equipment 60 (e.g., capital equipment). Medical device 10 may include a ureteroscope, endoscope, bronchoscope, duodenoscope, colonoscope, etc., which may include a shaft 30 that extends distally from a handle 20. As discussed below, medical device 10 may have two or more working channels and does not include a light source.

As explained below, medical system 100 may be used to treat medical issues, such as kidney stones. In an example, shaft 30 is introduced into a body lumen, such as a ureter, and advanced through the urethra until a distal portion of shaft 30 is located in a calyx of a kidney, for example, adjacent one or more kidney stones. Medical device 10 may include one or more working channels (e.g., through shaft 30), which enables multiple instruments to be used simultaneously within a lumen of the body.

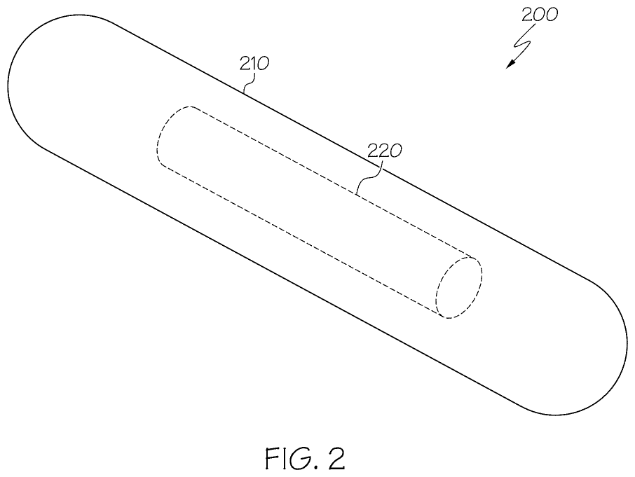

Medical system 100 further includes a first instrument 101, which may be used to control an expandable device such as a basket or a snare. As discussed in detail below, first instrument 101 may include an expandable basket or retrieval device. The expandable basket or retrieval device may be used, for instance, to place, position, and/or remove light source 200 (e.g., as depicted in FIG. 2) in a lumen of the body and/or to remove debris, such as a small kidney stone, a fragmented kidney stone, and/or one or more other objects, from the body of the subject.

Medical device 10 includes handle 20 with at least one actuator and at least one port. For example, medical device 10 may include, e.g., a first actuator 22 and a second actuator 24, and medical device 10 may include a first port 27 and a second port 28. Medical device 10 also includes shaft 30 with a steerable or deflectable portion 32 and a distal portion 30D. Actuators 22 and 24 may receive user input and transmit the user input to shaft 30. Each actuator 22 and 24 may include a lever, knob, slider, joystick, button, or other suitable control mechanism.

In an example, first actuator 22 may be configured to articulate steerable portion 32, e.g., via one or more pull wires (not shown) within shaft 30, and second actuator 24 may be configured to actuate and/or control other aspects of medical device 10, e.g., turning on/off laser sources, capturing video and/or images, etc. In some cases, equipment 60 may be configured to supply medical device 10 with vacuum/suction, one or more fluids (e.g., liquid, air, etc.), and/or power via umbilicus 26.

As depicted in FIG. 1A, equipment 60 may include a processor 62, e.g., operable with medical device 10. For example, processor 62 may help generate a visual representation of video and/or image data and/or transmit the visual representation to one or more interface devices, e.g., a display 12. According to some aspects, processor 62 may augment the visual representation. Display 12 may include, e.g., a touch-screen display or other display, capable of displaying images, optionally with information regarding features shown in the image(s), generated using medical device 10 and processor 62. While FIG. 1A shows processor 62 as part of equipment 60 coupled to medical device 10, additionally or alternatively, medical device 10 may include processor 62. For example, processor 62 may be included in handle 20 of medical device 10.

Port 27 on handle 20 may include a first opening in communication with a first working channel 34 (as depicted in FIG. 1B) of shaft 30. Similarly, port 28 on handle 20 may include a second opening in communication with a second working channel 35 of shaft 30. An instrument such as, e.g., a fiber (e.g., laser fiber), a grasper, a retrieval device, etc., may be inserted through a port, e.g., port 27 or port 28, and moved distally through shaft 30 to a corresponding working channel, and ultimately extended distal to shaft 30.

For example, first working channel 34 may be fluidly connected to and correspond to port 27, and second working channel 35 may be fluidly connected to and correspond to working port 28. By using first working channel 34 and second working channel 35, two instruments may be used simultaneously. For instance, a basket instrument may be used in first working channel 34, and a laser instrument may be used in second working channel 35. Other examples are possible.

One or more alternative instruments (e.g., a guidewire, a basket, a snare, forceps, etc.) may be used. Shaft 30 may further include one or more lumen(s) for receiving pull wires and/or other wiring, cables, and/or fluidics tubing. In one example, an instrument may be delivered through one of first working channel 34 or second working channel 35. Additionally, suction or negative pressure may be applied, or one or more fluids may be delivered through the other of first working channel 34 or second working channel 35.

As depicted in FIG. 1B, distal portion 30D of shaft 30 may include a distal opening of the first working channel 34, a distal opening of the second working channel 35, and an imager 42 (e.g., camera or other imaging device). In some aspects, distal portion 30D may also include a sensor 46. Examples of sensor 46 include, but are not limited to, a pressure sensor, a distance sensor, a light sensor, a temperature sensor, an ultrasonic sensor, etc.

In some examples, shaft 30 may include an end cap 33 covering portions of distal portion 30D, e.g., protecting features such as imager 42 and/or sensor 46. In some examples, imager 42 may include a camera, for example, including a Complementary metal-oxide semiconductor (CMOS) sensor. In some examples, imager 42 may include fiber optics in communication with a sensor or other device within shaft 30 or handle 20. For example, although not shown, shaft 30 and/or handle 20 may include one or more wires, cables, etc. connecting imager 42 to one or more processors (e.g., in equipment 60 and connected via umbilicus 26).

First instrument 101 may include a sheath 109 and/or a shaft 110. Sheath 109 and shaft 110 are positioned on a distal portion of first instrument 101. Shaft 110 is connected to end effector 102. First instrument 101 is configurable to insert, remove, open, close, and otherwise configure end effector 102. As discussed herein, end effector 102 may be used to insert or remove objects into a lumen of the body and/or to remove objects such as kidney stones from the body.

For example, end effector 102 may be configured to grasp and retain a light source such as light source 160 (or light source 200 depicted in FIG. 2). As further discussed herein, light source 160 may be held in arms 116 and positioned just distal to distal portion 30D such that the light source aids in positioning of the distal end of the shaft within a lumen of a body.

First instrument 101 may include a handle 105, an actuator 107 movably coupled to handle 105, and a plunger 108 that is movably mounted to actuator 107. Sheath 109 and/or shaft 110 may be attached to plunger 108. In one example, shaft 110 is attached to a distal portion of end effector 102. In another example, a wire (not depicted) may extend distally through handle 105, actuator 107 and plunger 108 from handle 105. Shaft 110 may be attached to a distal portion of the wire. End effector 102 may be attached to a distal portion of either the wire or the shaft. The wire may be an elongated metallic element, such as a surgical wire, although any type of material may be used. As depicted, sheath 109 and/or shaft 110 are inserted into or coupled with port 28, but shaft 110 may also be inserted into or coupled with port 27.

In one example, actuator 107 may be operable to extend shaft 110 and end effector 102 distally from sheath 109 to surround a stone or other material, and retract end effector 102 proximally into sheath 109 to capture the object or other material. Specifically, actuator 107 may slide relative to handle body 106 in a proximal or distal direction to move the wire and therefore shaft 110 proximally or distally through sheath 109. In these aspects, movement of actuator 107 may deploy end effector 102 out from sheath 109 (in a deployed configuration) or retract end effector 102 into sheath 109 (in a retracted configuration).

In another example, actuator 107 may be configured to move one or more of the wire, shaft 110, and/or sheath 109 in a proximal-distal direction along (e.g., parallel to or coaxial with) longitudinal axis 199. Actuator 107 may be positioned within channel 188 and may be configured to slide within channel 188. Actuator 107 may have a distal or closed position where sheath 109 is positioned over end effector 102, and a proximal or open position where sheath 109 is positioned proximal to end effector 102 and end effector 102 is deployed outside of sheath 109. Actuator 107 may be configured to move a set distance within channel 188 between the distal or closed position and the proximal or open position. In some examples, as discussed below, actuator 107 may be spring-biased towards either the distal or closed position or the proximal or open position.

Any type of end effector 102 may be used, including any closed-ended configurations (e.g., a basket) or open-ended configurations (e.g., two or more opposing jaws). Non-limiting examples of end effector 102 include a basket, a grasper, a snare, and a snare net. End effector 102 is depicted as a closed-ended basket made of a shape memory metal (e.g., Nitinol). End effector 102 may include one or more arms 116 extending radially-outward, relative to central longitudinal axis 199, from shaft 110. The arms 116 may be evenly spaced around a circumference of shaft 110. Each arm 116 may be coupled to each other at a distal portion 120 of end effector 102.

End effector 102 may also be an open-ended grasper, or like capture device. Accordingly, end effector 102 may be operable between a closed position, in which end effector 102 is collapsed inside of sheath 109, and an open position, in which end effector 102 automatically expands after being extended distally out of sheath 109.

As further explained below, end effector 102 may be deployed with each arm 116 positioned at least partially distal from shaft 110 and/or sheath 109. Alternatively, end effector 102 may be in a retracted configuration with each arm 116 positioned entirely within and covered by sheath 109. Each arm 116 may expand radially-outward when transitioned from the retracted configuration to the expanded configuration.

Handle 105 may be grasped in a hand of or otherwise manipulated by a user and/or attached to another medical device. Handle 105 may include a body 106 extending along a central longitudinal axis 199 of first instrument 101. Body 106 is depicted as having a cylindrical shape, although any shape may be used. The exterior surfaces of body 106 may be contoured to define a grip surface. A channel 188 may extend through body 106 parallel to longitudinal axis 199. Plunger 108 may be movably mounted to actuator 107 and configured to enable rotation of sheath 109.

FIG. 2 depicts an exemplary light source 200, according to aspects of this disclosure. Light source 200 may be oblong or pill-shaped, having a diameter (e.g., a lateral diameter) that is less than a diameter of one or more of working channels 34 and 35.

Light source 200 may be activated and secured within end effector 102. End effector 102 may be then inserted into medical device 10 and positioned at or adjacent to distal portion 30D. Subsequently, the shaft 30 of medical system 100 may be introduced through a lumen of the body, for example, a urethra. Light source 200 may help illuminate a portion of a lumen of a subject (e.g., urinary tract) to aid in visual navigation of distal portion 30D to a treatment site. This helps to allow for more precise navigation to the targeted location. Upon reaching the desired location, light source 200 may be released from the end effector 102 and positioned appropriately to help provide visibility for a subsequent medical procedure.

Light source 200 includes an outer tube 210 and an inner vial 220. In some examples, the outer tube 210 is formed of a polycarbonate plastic, and inner vial 220 is formed a material that can be broken when bent, for example, glass or another brittle or breakable material. Outer tube 210 may include a first substance, such as hydrogen peroxide, in addition to inner vial 220. Inner vial 220 may contain a second substance, such as phenyl oxalate ester with fluorescent dye.

In an example, outer tube 210 is bent or otherwise manipulated by an external force, for example, applied by a physician. The external force breaks inner vial 220, thereby mixing the two solutions. As the chemicals react, they release energy in the form of visible light. Additionally, outer tube 210 may serve to protect the contents of light source 200, including inner vial 220.

A diameter and size of light source 200 is limited by a diameter of the working channels 34 and 35 of medical device 10. Light source 200 may be manufactured in different sizes for different procedures and equipment. In an example, a working channel has a diameter of Ø0.046 inch (Ø1.168 mm). In this scenario, light source 200 may have a maximum outer diameter about Ø0.045 inch (Ø1.143 mm). A length of light source 200 may be mainly determined by anatomy. Additionally, secondary concerns may be considered, such as the bending radius of the ureteroscope, the geometry of the baskets available for maneuvering the light source, etc.

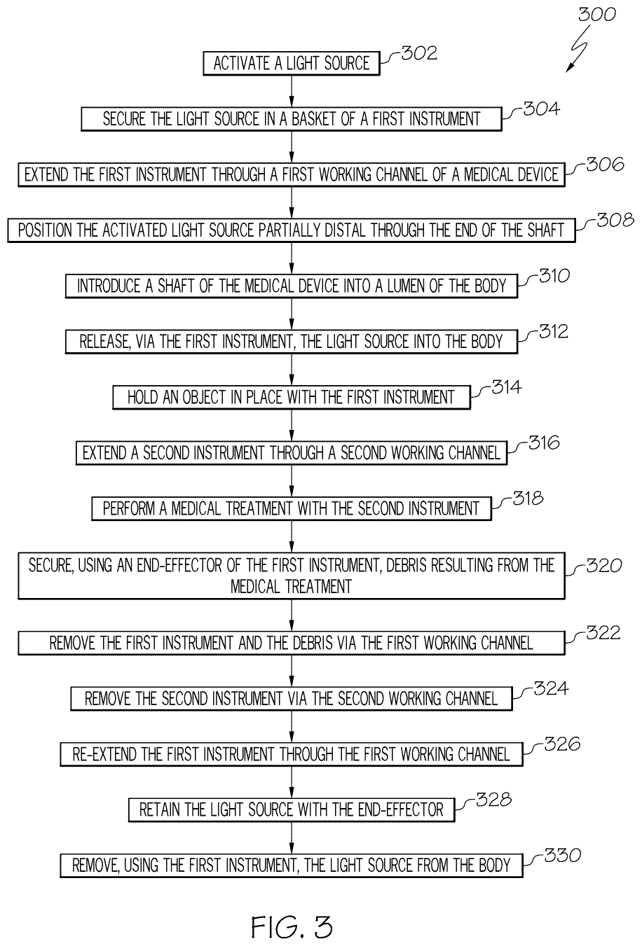

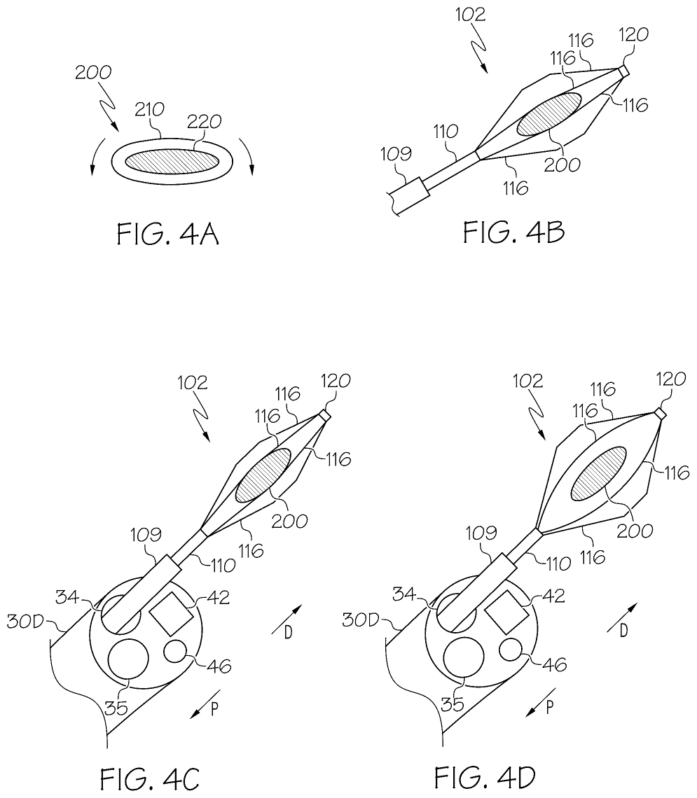

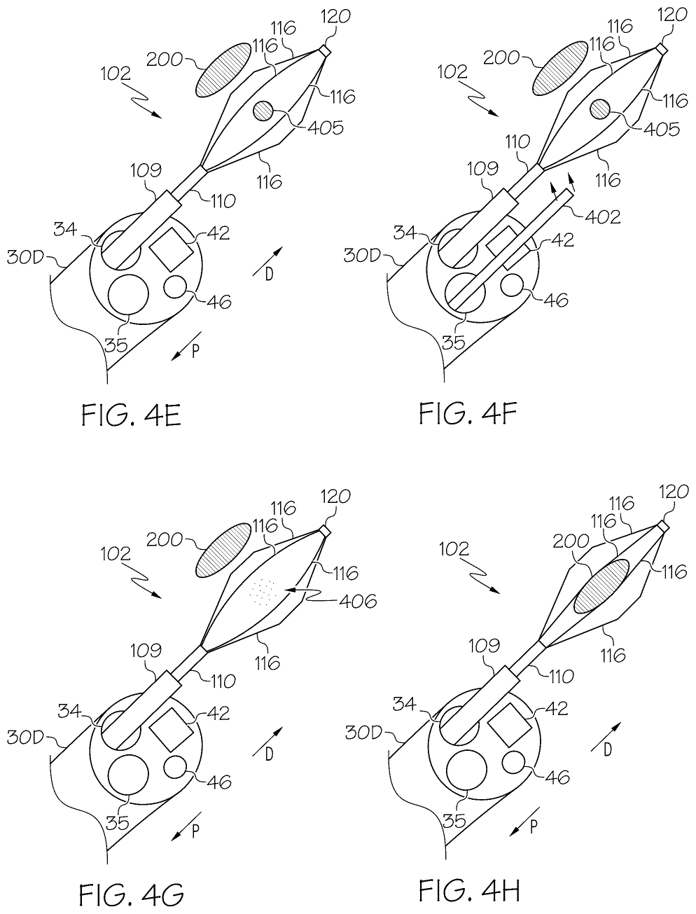

FIG. 3 depicts a flow chart of an exemplary method 300 for performing a medical treatment, according to aspects of this disclosure. Method 300 may be performed with any of the medical systems or devices discussed herein (e.g., medical system 100, medical device 10, first instrument 101, and/or lighting source 200). Method 300 describes various blocks or steps, each of which include one or more operations. The blocks and/or the operations are described in an exemplary order for discussion purposes. In some cases, one or more blocks or operations may be performed out-of-order, and/or one or more blocks or operations may be skipped or otherwise omitted from method 300. Method 300 may be performed by a user or operator, for example, a physician or technician, of the medical systems or devices disclosed herein. FIG. 3 is discussed in conjunction with FIGS. 4A-4H.

FIGS. 4A-4H depict views of operation of an exemplary medical system, according to aspects of this disclosure. Various aspects shown in FIGS. 4A-4H illustrate various features of method 300. For instance, FIG. 4A depicts light source 200, with outer tube 210 and inner vial 220. Arrows indicate direction in which force is applied, e.g., by hand, to break inner vial 220. Light source 200 is activated and may then be used in conjunction with a medical procedure.

At block 302, method 300 may involve activating a light source. For example, a user, e.g., a physician, activates light source 200 by bending outer tube 210, thereby breaking inner vial 220. The chemicals within outer tube 210 and inner vial 220 mix, react, and cause light to be emitted.

At block 304, method 300 may involve securing the light source in a basket of a first instrument. An example of first instrument 101 is a basket instrument. First instrument 101 includes end effector 102 attached at a distal portion of shaft or sheath 109. As discussed, end effector 102 may be expandable such that end effector 102 may be used to grasp objects for insertion or removal into a lumen of the body. In some cases, the operations of blocks 302 and 304 can be performed together.

For instance, end effector 102 may be used to releasably retain light source 200. For example, a user may position light source 200 within a basket, for example, formed by arms 116 of end effector 102. End effector 102 releasably retains light source 200 while the light source 200 is extended through first working channel 34 of medical device 10.

FIG. 4B depicts end effector 102, including arms 116, sheath 109, shaft 110, and distal portion 120 of end effector 102; and light source 200. As depicted, light source 200 is positioned within arms 116 of the basket of end effector 102.

Returning to FIG. 3, at block 306, method 300 may involve extending the first instrument through a first working channel of a medical device. Continuing the example, first instrument 101, including end effector 102 retaining light source 200, may be inserted into port 28 and extended through first working channel 34 towards to distal portion 30D of shaft 30.

At block 308, method 300 may involve positioning the activated light source at least partially distal relative to the end of the shaft. In some cases, the activated light source 200 may be positioned, using end effector 102, through distal portion 30D of shaft 30 and extending partially distal to the distal portion 30D of the shaft 30 and into the body. This approach is depicted in FIG. 4C. The positioning may occur prior to releasing the activated light source 200 into the body.

FIGS. 4C-4H depict distal portion 30D of shaft 30, including distal opening of first working channel 34, distal opening of working channel 35, imager 42, and sensor 46 and end effector 102 including sheath 109 and shaft 110 of end effector 102, arms 116, and distal portion 120. FIG. 4C further depicts arms 116 of end effector 102 in a closed configuration, for example, in a closed basket configuration, such that arms 116 are grasping or otherwise securing light source 200.

In some cases, positioning the activated light source may be accomplished by using the relative movement of shaft 110 and sheath 109. For instance, the light source may be positioned and then the shaft 110 and/or sheath 109 repositioned, thereby enabling additional operations, without adjusting the shaft of the first instrument.

This approach secures the light source 200 and minimizes movement of the light source 200 relative to the distal portion 30D of the shaft 30. In this manner, the light source 200 may help to illuminate the area adjacent to the distal portion 30D, for example, to help guide the placement of shaft 30 within the body, for instance, proximate to a treatment site.

Returning to FIG. 3, at block 310, method 300 may involve introducing a shaft of the medical device into a lumen of the body. As discussed above with respect to block 306, light source 200 may guide placement through the lumen of the body.

For example, the shaft 30 may be introduced through the urethra, with the light source 200 held firmly in end effector 102 illuminating the path, allowing for precise navigation to the targeted location, for example, proximate to a kidney stone or other object that needs treatment such as ablation and/or removal. The secured light source 200 guides placement of shaft 30D.

Distal portion 30D (and thus imager 42) of medical device 10 may be positioned proximate a target object. The target object may be various anatomical or foreign objects, such as a kidney stone.

At block 312, method 300 may involve releasing, via the first instrument, the light source into the body. Continuing the example, upon reaching the desired location, light source 200 may be released from the end effector 102 and positioned appropriately to optimize visibility for the ensuing medical procedure. FIG. 4D depicts such a configuration.

FIG. 4D depicts sheath 109 and shaft 110 of end effector 102, with arms 116 and distal portion 120. Arms 116 are opened to release light source 200. To open arms 116, for example, sheath 109 may be proximally retracted relative to shaft 110 to transition arms 116 to the open configuration. Alternatively or additionally, shaft 110 may be extended distally relative to sheath 109 to transition arms 116 to the open configuration. Then, light source 200 may be released from arms 116 (FIG. 4E) to be positioned at or adjacent to the treatment site to help illuminate the treatment site.

Accordingly, shaft 110 and sheath 109 may move relative to each other to facilitate releasing the light source.

In some cases, end effector 102 may be removed or retracted proximally through the working channel, either partially or fully. Removing the end effector 102 may provide additional working area for the medical procedure. If so, then the first instrument, including end effector 102, may be re-inserted distally through first working channel 34 at a later or other portion of the procedure.

At block 314, method 300 may involve holding an object in place with the first instrument. Continuing the example, when the end effector 102 is extended distally from distal portion 30D of the shaft 30, then end effector 102 may be configured to grasp and/or hold object 405 in place. For example, object 405 may be kidney stone that is targeted for ablation. This configuration is depicted in FIG. 4E, which depicts light source 200 placed in a lumen of the body, and arms 116 surrounding object 405.

Returning to FIG. 3, at block 316, method 300 may involve extending a second instrument through a second working channel. Examples of a second working instrument include, but are not limited to, an ablation tool, a laser fiber, a knife, a stapler, and an electrode. Continuing the example, a second instrument is introduced at the proximal end of the working channel 35 and extended distally through the working channel 35. This is depicted in FIG. 4F.

FIG. 4F depicts light source 200 placed in the lumen of the body with arms 116 grasping or otherwise surrounding object 405. As mentioned above, relative movement of one or more of sheath 109 and/or shaft 110 may help to transition arms 116 from an open configuration to a closed configuration, and vice versa. A fiber 402 may be introduced and extended through distal portion of first working channel 34. Fiber 402 may be movable within first working channel 34 and may be extended distally through first working channel 34 such that at least a portion of fiber 402 exits first working channel 34 to be distal to distal portion 30D of medical device 10.

The fiber 402 may include a laser fiber, e.g., capable of fragmenting a stone via laser lithotripsy. Fiber 402 may include a transparent or translucent material, which may provide for the ability to visualize a portion of a target that is behind the fiber (e.g., otherwise obscured from view by an opaque fiber or by a sheath of fiber 402). In other aspects, the fiber may include an opaque material. Optionally, fiber 402 and/or the sheath (not shown) may include one or more markings, which may be indicative of a distance relative to distal portion 30D of medical device 10.

Returning to FIG. 3, at block 318, method 300 may involve performing a medical treatment with the second instrument, for example, on object 405. As depicted in FIG. 4F, a source of energy is emitted, e.g., via a laser source, to treat object 405.

At block 320, method 300 may involve securing, using an end effector of the first instrument, debris resulting from the medical treatment. The treatment (e.g., laser fragmentation) performed at block 318 may cause object 405 to fragment. The fragments are typically gathered for removal.

FIG. 4G depicts light source 200 placed in the lumen of the body with arms 116 grasping the debris 406. Continuing the example, end effector 102 may be reconfigured or replaced (e.g., with a different end effector) to grasp or otherwise secure debris 406 resulting from the medical treatment.

Returning to FIG. 3, at block 322, method 300 may involve removing the first instrument and the debris from via the first working channel. Continuing the example, end effector 102, which may be grasping or securing debris 406, may be removed from the treatment site through first working channel 34.

At block 324, method 300 may involve removing the second instrument via the second working channel. Continuing the example, fiber 402 is removed from first working channel 34.

At block 326, method 300 may involve re-extending the first instrument through the first working channel. At block 326, similar operations are performed as discussed with respect to block 306.

At block 328, method 300 may involve retaining the light source with the end effector. FIG. 4G depicts arms 116 grasping the debris created at block 322. End effector 102 closes, bringing arms 116 in a closed position. Then, end effector 102 is removed proximally through the working channel 35, thereby removing the debris.

At block 330, method 300 may involve removing, using the first instrument, the light source from the body. Removing the light source is depicted in FIG. 4H. FIG. 4H depicts arms 116 grasping or otherwise securing light source 200. End effector 102 closes, bringing arms 116 in a closed position. To close arms 116, sheath 109 is extended distally and/or shaft 110 is retracted proximally.

End effector 102 is then removed proximally through first working channel 34, thereby removing debris 406. The operations described with respect to 320-330 may ensure that all inserted elements are accounted for and safely removed, marking the completion of the process.

While principles of this disclosure are described herein with reference to illustrative aspects for particular medical uses and procedures, this is not limited thereto. Those having ordinary skill in the art and access to the teachings provided herein will recognize additional modifications, applications, aspects, and substitution of equivalents all fall in the scope of the aspects described herein. Accordingly, this disclosure is not to be considered as limited by the foregoing description.

1. A medical system comprising:
a medical device comprising:
a shaft defining a plurality of working channels, each working channel having a respective lumen with a respective distal opening, the distal openings positioned at a distal portion of the shaft; and
an imaging device positioned at the distal portion of the shaft;
an instrument movable through one or more of the working channels, wherein a distal portion of the instrument includes an expandable end effector; and
a light source, wherein the light source is releasably positionable within the expandable end effector of the instrument, and
wherein the medical device does not comprise a light source.

2. The medical system of claim 1, wherein the medical device further comprises a handle positioned on a proximal end of the shaft, the handle comprising a port having a corresponding working channel of the plurality of working channels.

3. The medical system of claim 1, wherein the instrument does not comprise an imaging device.

4. The medical system of claim 1, wherein the medical device is a ureteroscope.

5. The medical system of claim 1, wherein the expandable end effector comprises an expandable basket.

6. The medical system of claim 1, wherein the light source comprises an outer tube and an inner vial, wherein the outer tube contains a first substance, and the inner vial contains a second substance, and wherein, when mixed, the first substance and the second substance cause a chemical reaction that causes a light to be emitted.

7. The medical system of claim 6, wherein the outer tube is formed of polycarbonate plastic, and wherein the inner vial is formed of glass.

8. The medical system of claim 1, wherein the light source comprises a diameter less than a diameter of at least one of the plurality of working channels.

9. The medical system of claim 1, wherein a first working channel of the plurality of working channels comprises a first diameter, and wherein a second working channel of the plurality of working channels comprises a second diameter that is greater than the first diameter.

10. The medical system of claim 9, wherein the first diameter is within a range of 0.02 to 0.04 inches, and wherein the second diameter is within a range of 0.05 to 0.07 inches.

11. The medical system of claim 9, wherein the first diameter is 0.03 inches, and wherein the second diameter is 0.06 inches.

12. The medical system of claim 1, wherein the medical device further comprises a pressure sensor positioned on the distal portion of the shaft.

13. The medical system of claim 1, wherein the expandable end effector is configurable to releasably retain the light source and position the light source within a lumen of a body.

14. The medical system of claim 1, further comprising an additional instrument that is movable through an additional working channel of the plurality of working channels.

15. The medical system of claim 14, wherein the additional instrument is a laser fiber.

16. A method of treatment of a body of a subject, the method comprising:
activating a light source;
securing the light source within an expandable end effector of a first instrument, wherein the expandable end effector is positioned at a distal portion of the first instrument and is configured to releasably retain the light source;
extending a distal portion of the first instrument through a first working channel of a medical device, the medical device comprising a shaft, wherein the medical device includes the first working channel and a second working channel each extending longitudinally through the shaft;
introducing the shaft into a lumen of the body;
releasing, via the expandable end effector, the light source into the body; and
extending a second instrument through the second working channel.

17. The method of claim 16, wherein the expandable end effector is an expandable basket, the method further comprising, prior to releasing the light source into the body, positioning, with the expandable end effector of the first instrument, the light source into the body by partially extending the first instrument distal to the distal portion of the shaft.

18. The method of claim 16, further comprising:
performing a medical treatment with the second instrument;
securing, using the end effector of the first instrument, debris resulting from the medical treatment;
removing, using the first instrument, the debris via the first working channel; and
retrieving, using the first instrument, the light source from the lumen of the body via the first working channel.

19. A method of treatment of a body of a subject, the method comprising:
activating a light source, wherein the light source comprises an outer tube and an inner vial, wherein the outer tube and the inner vial contain substances that mix to emit light;
securing the light source within an expandable end effector of a first instrument;
introducing a shaft of a medical device into and through a lumen of the body, wherein the medical device includes at least a first working channel and a second working channel extending longitudinally through the shaft, and wherein the medical device includes an imaging device at a distal portion of the shaft;
extending the first instrument through the first working channel of the medical device, wherein the first instrument is configured to releasably retain the activated light source; and
positioning, with the expandable end effector, the activated light source into the body by partially extending the first instrument partially distal to the distal portion of the shaft.

20. The method of claim 19, further comprising:
removing the first instrument at least partially through the first working channel;
re-inserting the first instrument via the first working channel;
capturing the light source with the expandable end effector; and
removing the first instrument and the light source via the first working channel.
